(19) United States
(12) Patent Application Publication
(10) Pub. No.: US 2012/0271350 A1
(43) Pub. Date: Oct. 25, 2012

(54) Range of motion table

(75) Inventor: Brent Warren Palmer, St Louis Park, MN (US)

(73) Assignee: ROM-T Enterprises LLC, St Louis Park, MN (US)

(21) Appl. No.: 13/064,874

(22) Filed: Apr. 22, 2011

(51) Int. Cl. A61F 5/00

(52) U.S. Cl. 606/242

(57) ABSTRACT

A massage table for supporting a person during a massage above a surface includes a leg assembly that engages the floor, and a table top that includes six foldable wings, four staging feet and a handle. The frame is made up of rectangular aluminum sections that are welded together to make up the frame and the wings frame. The frame includes a frame base that supports at least a portion of the person above the surface with two legs, the wings are connected to the base of the frame by piano hinge and can fold down independently while still connected to the frame base, the frame sections are made of aluminum but can be made up of various materials. A T-bar face cradle inserts into the head of the table.

### BACKGROUND AND SUMMARY OF INVENTION

Massage therapy is becoming a large part of America's health. The ROM-T (Range of Motion Table) was invented to make it possible to perform range of motion on a client without the table being in the way thus interrupting the client's therapy because the therapist has to ask the client to move to the edge of the table. The table is a portable table with two halves hinged in the center to allow for collapsing the two halves and latched by a typical latch that holds the two halves together. The face cradle can be stored in the table or in the pocket on the bag.

The ROM-T features wings, these wings fold down still connected to the table by a piano hinge. While the wings of the table are folded down it allows the therapist access to three hundred and sixty degrees range of motion. There are six wings that fold down independently or as a group, the middle two wings are square in shape and the top wings are cut at an angle so the wing angles into the area of the scapula. In the folded down position the therapist is able to use the table for leverage by moving into the table improving their center of gravity which leads to better body mechanics. Both legs fold down and into the table, there are no removable parts on our table and the frame is half moon shaped to accommodate the A frame foot. The A frame leg is in the shape of an A that connects to the frame and proceeds to the floor, it has a push button height adjustment in the leg. ROM-T is a versatile table that will also benefit the Chiropractic and Physical Therapy industries with its innovation. Current massage tables on the market do not offer the ability for three hundred sixty degree range of motion without asking the client to move; asking the client to move takes the client out of their relaxed state of mind and causes them to feel uncomfortable when resting on the edge of the table.

### BRIEF DESCRIPTION OF THE FIGURES

We added detailed drawings marked as FIGS. 1 thru 12 to pending Provisional Application Ser 61/343,245

FIG. 1 shows the folded state of the massage table for transport or storage including, handle, staging feet and piano hinge side;

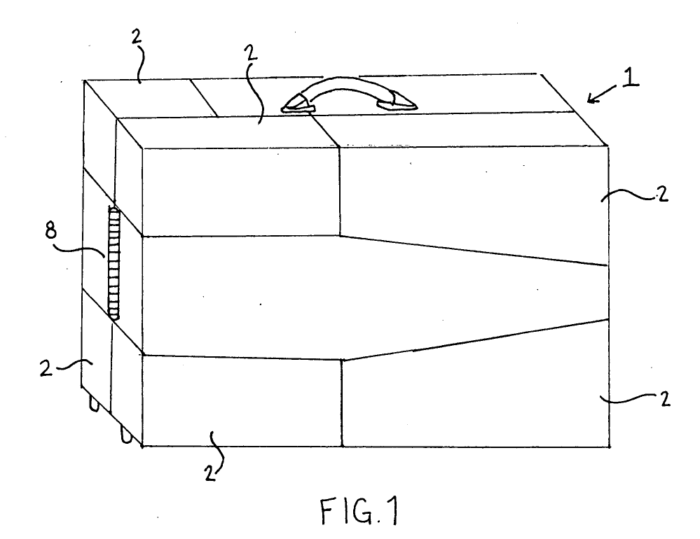

FIG. 2 shows folded state of ROM-T from the head side with handle, staging feet, latches and orifice for the tri positional face cradle mount;

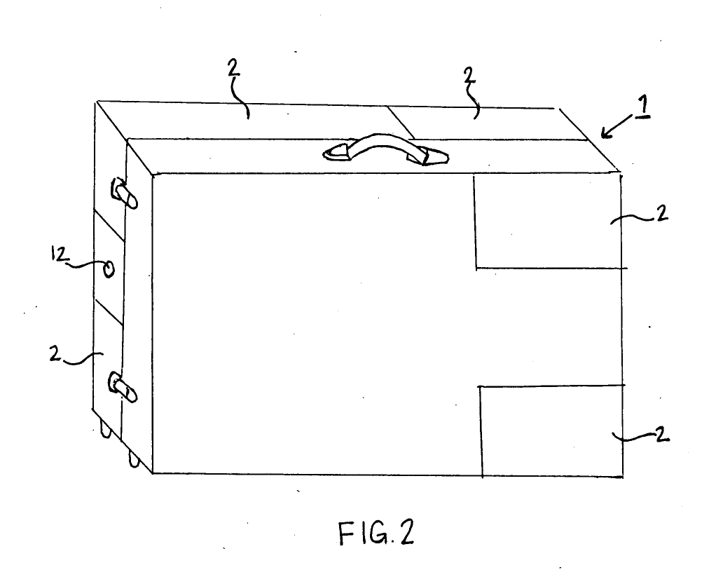

FIG. 3 upright working position with legs extended down; face cradle orifice and wings on each side of the table. Center line defines where the ROM-T folds in half for portability and storage;

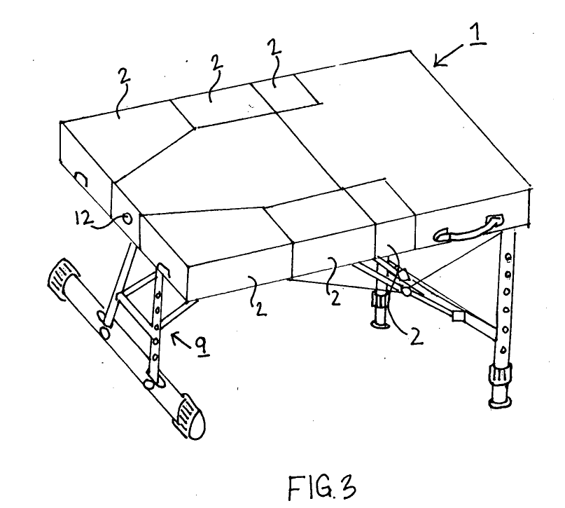

FIG. 4 reverse view similar to FIG. 3 with rear leg;

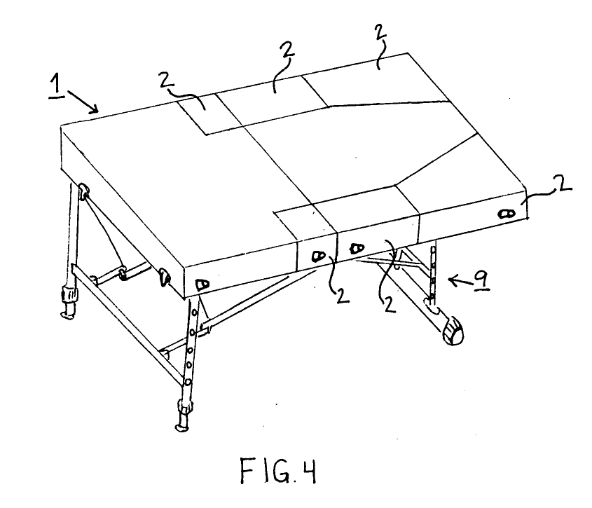

FIG. 5 top view of ROM-T showing front wing in the folded position attached by piano hinge and two orifices for the T bar to slide through and secure the wing to the frame;

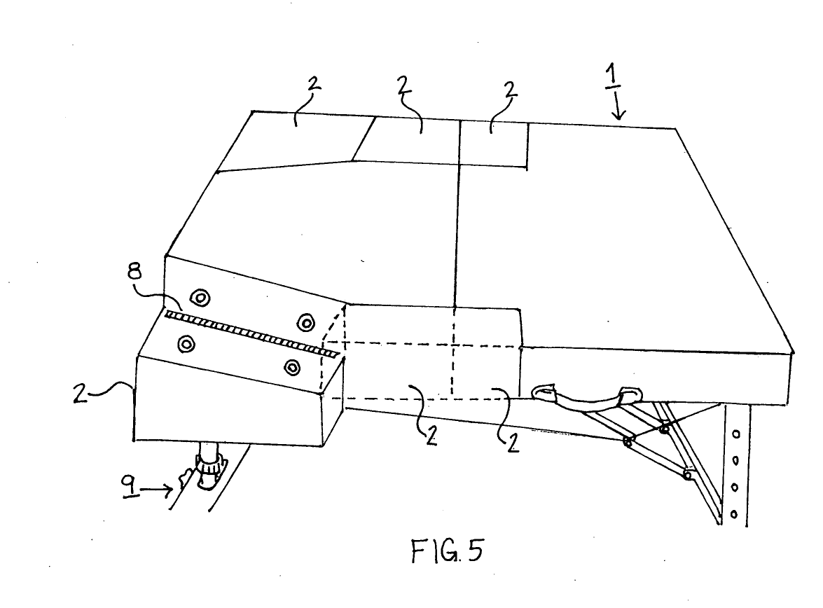

FIG. 6 top view of ROM-T with the middle and lower wings in the folded position attached by piano hinge and each wing has an orifice for the T bar to slide into and secure the wing to the frame. The middle wing cut in a half moon shape to allow the front foot to reside;

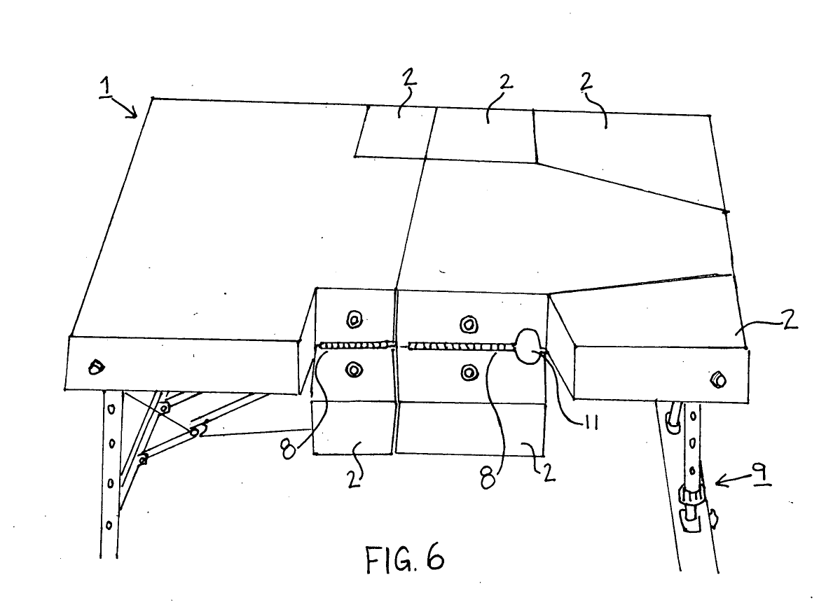

FIG. 7 front leg assembly;

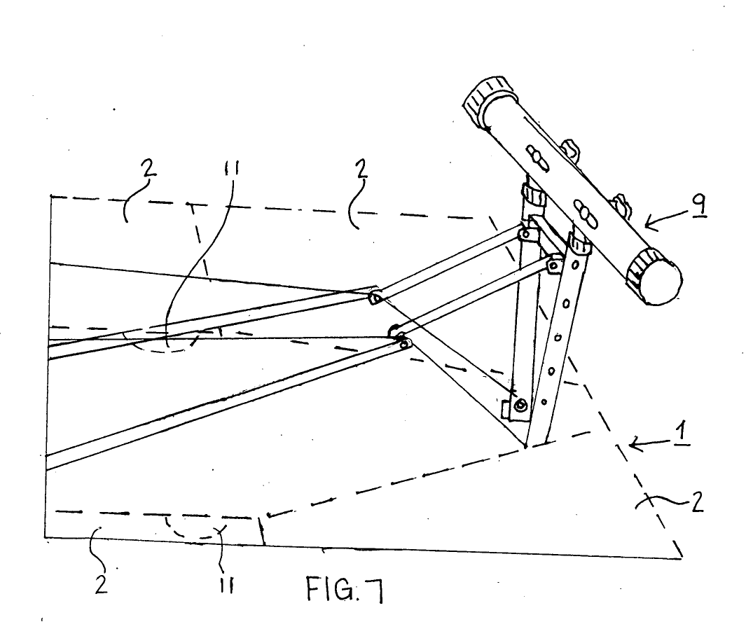

FIG. 8 underside view of ROM-T showing the T bar stops, T bars, T bar guides, piano hinges, T bar face cradle mount, half moon cut out and wings;

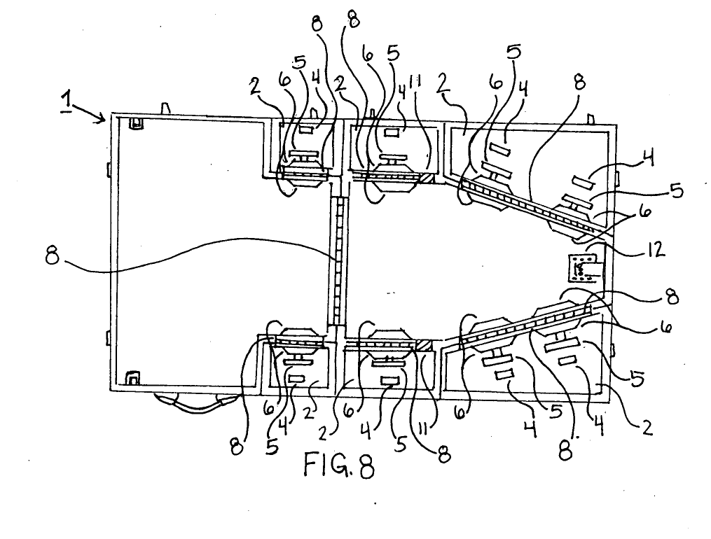

FIG. 9 Top view defining the wings location, staging feet and handle;

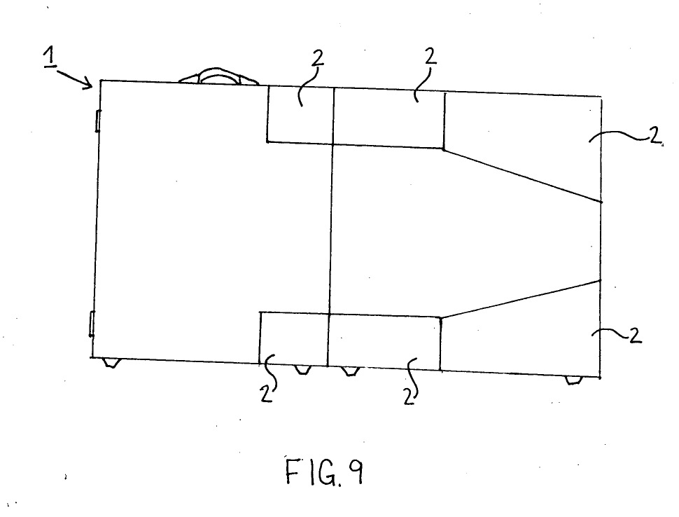

FIG. 10 T bar latch with T bar guide on each side of the frame, T bar stop;

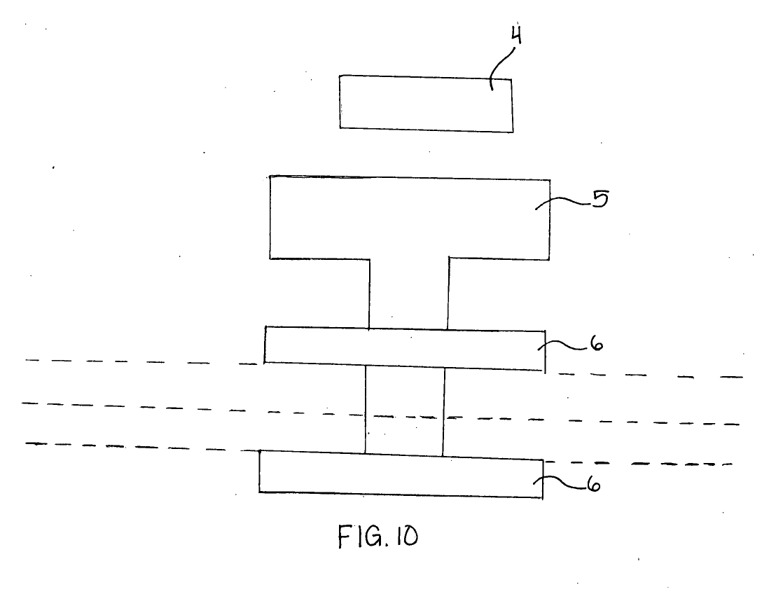

FIG. 11 bottom view of tri positional face cradle with T Bar;

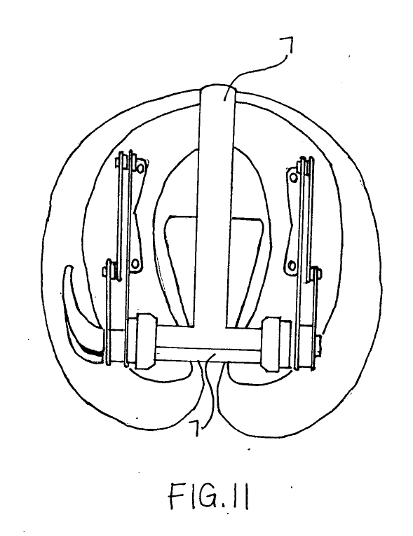

FIG. 12 Tri positional face cradle T bar assembly in the open position;

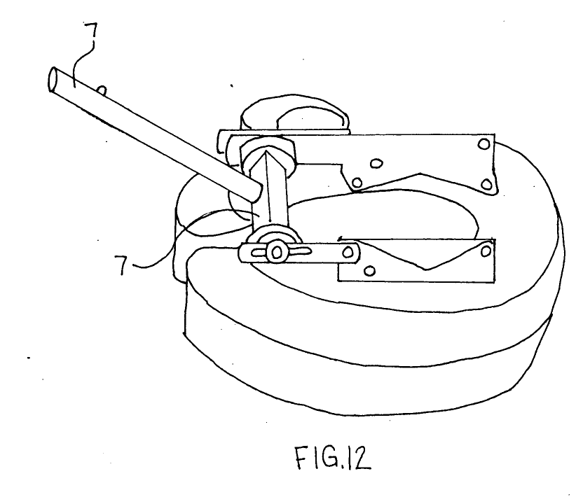

### DESCRIPTION OF THE INVENTION

ROM-T massage table (1) with several unique features defined as: wings (2), T bar stops (4),T bar (5), T bar guides (6) Tri positional face cradle T bar (7), Hinges (8), A frame leg (9), half moon cut out (front leg storage) (11), Tri positional face cradle mount (12). Please refer to the claims for additional information and descriptions.

1. A massage table for supporting a person during a massage above a surface includes a leg assembly that engages the floor, and a table top that includes six foldable wings, four staging feet and a handle. The frame is made up of rectangular aluminum sections that are welded together to make up the frame and the wings frame. The frame includes a frame base that supports at least a portion of the person above the surface with two legs, the wings are connected to the base of the frame by piano hinges and can fold down independently while still connected to the frame base, the frame sections are made of aluminum but can be made up of various materials. A T-bar face cradle inserts into the superior transverse portion of the table.

2. The table of claim 1 wherein the massage table is made of various materials but not limited to: aluminum, wood and/or any other natural or manufactured material.

3. The table claim of 2 wherein the massage table with six foldable wings three on each side connected by piano hinge to top and bottom parts of table. The wings fold down independently allowing the therapist to provide three hundred and sixty degree rotation in the shoulder and hip regions. While the wings are in the folded down position, the wings allow the therapist to lean into the massage table providing additional support for the therapist using the massage table for leverage and improved center of gravity thus improving the therapist's body mechanics. In the frame and the wing design there are specific cut away half moon shaped voids to accommodate the front A frame foot for storage purposes.

4. The table claim of 3 wherein the massage tables wings each have a T-bar latch that push in from the wing to the frame to support the wing. The latch is made up of four pieces: the T- bar, T-bar stop that is connected securely to the underside of the wing that stops the T- bar from pulling all the way out of the wing as defined in claim 3, each T- bar latch runs through a T-bar guide located on both the frame and the wing.

5. The table of claim 4 wherein the A frame leg is connected to the proximal underside of the frame using a bolt, nut and washers to welded stationary support. The front leg is in the shape of an A because the middle portion of the top section is cut at an angle to accommodate the scapula region. The leg is specially designed for that purpose. The front and the rear legs are reinforced with typical cables used on massage tables today.

6. The table of claim 5 wherein the tri-positional face cradle T-bar rotates and locks in the left, center and right position with a spring loaded button. The face cradle mounts securely to the proximal portion of the underside of the table. The mount is long enough to accommodate the face cradle T-bar and has three holes drilled for locking left, right and center.

7. The table of claim 6 wherein the massage table has six piano hinges that secure the wings to the frame of the table allowing the wings to fold down still attached to the table. One piano hinge attached to the center of the table allowing it to fold in half.
